(19) United States
(12) Patent Application Publication
(10) Pub. No.: US 2007/0248696 A1
(43) Pub. Date: Oct. 25, 2007

(54) Composition and method for enhancing neuromuscular facilitation and cognitive functions

(75) Inventors: Pete Maletto, Long Branch, NJ (US); Daniel Maiullo, Tinton Falls, NJ (US)

Correspondence Address: CATALINA + ASSOCIATES, LLC, 167 AVENUE AT THE COMMON, SUITE 9, SECOND FLOOR, SHREWSBURY, NJ, 07702, US

(73) Assignee: Mind Sports Nutrition Inc.

(21) Appl. No.: 11/410,490

(22) Filed: Apr. 25, 2006

(51) Int. Cl. A61K 36/906; A61K 36/82; A61K 31/7072; A61K 31/14; A61K 31/13

(52) U.S. Cl. 424/729; 424/756; 514/49; 514/642; 514/667

(57) ABSTRACT

A composition and a method for improving neuromuscular facilitation, also known as “muscle memory,” and enhancing cognitive functions, such as memory and mental focus. The dietary supplement, and the method for the administration thereof, increases acetylcholine levels, which improves neuromuscular facilitation. In various embodiments, factors other than increasing acetylcholine levels within the body further contribute to the overall effect of the dietary supplement composition. In a preferred embodiment, the invention is a composition, and a method for the administration thereof, comprising effective amounts of choline, dimethylaminoethanol, cytidine 5′-diphosphocholine, turmeric extract, decaffeinated green tea extract, vitamin B₁, vitamin B₅, vitamin B₆, vitamin B₁₂, folic acid, dimethylglycine, huperzine A, Griffonia simplicifolia extract, and 1-phenylalanine, magnesium stearate, and silicon dioxide.

### FIELD OF THE INVENTION

The present invention, with its various embodiments, generally relates to a dietary nutritional supplement composition, and a method for the administration thereof, for improving neuromuscular facilitation, also known as “muscle memory,” and enhancing cognitive functions, such as memory and mental focus.

### BACKGROUND AND SUMMARY OF THE INVENTION

When an active person trains movement—i.e., repetitively performs certain physical movements—in an effort to stimulate the body's adaptation process, the end result is increased levels of accuracy in performing those physical movements. That is, as the body assimilates a given physical activity and adapts to the training, the body's ability to “remember” a given exercise, repetition after repetition, induces its own form of “muscle memory.” “Muscle memory” is a common term for neuromuscular facilitation, which is the process of the neuromuscular system assimilating a given physical activity and adapting to the training, thereby improving fine and gross motor skills. Fine motor skills are very minute and small skills we perform with our hands as brushing our teeth, combing our hair, or using a pencil or pen to write. Gross motor skills are those actions that require large body parts and large body movements like throwing a baseball and swimming. There are certain physical activities that require high levels of both fine and gross motor skills, such as golfing and archery. Since the term “muscle memory” is synonymous with “neuromuscular facilitation,” these two terms will be used interchangeably throughout the discussion of the present invention.

Muscle memory is achieved over time through repetition of a given physical activity and the body's ability to assimilate and adapt to that training. For example, when one first begins to learn how to drive a vehicle, the novice driver quickly realizes it is not as easy as it looks. As the student driver reinforces the movements involved with driving a vehicle day after day after day, the driver's neuromuscular system learns the fine and gross motor skills to the degree that he or she is no longer required to think about them, but merely react and perform. Eventually, when the driver sits in the driver's seat, the driver automatically has a certain motion, style, amount of pressure exerted on the gas pedal, etc., as the individual drives the car without thinking about each movement. It just happens. This is muscle memory. The concept of muscle memory is widely recognized in the athletic fields that require intense mental focus and great accuracy in performing repetitive physical movements, such as golfing, archery, racing a car, throwing a football, and so on.

As the name suggests, “neuromuscular facilitation” involves transmission of information from one neuron (i.e., a nerve cell) to a muscle, resulting in muscular contraction. This transmission of information is accomplished by neurotransmitters. As such, neurotransmitters are an integral part of the present invention. That is, the various dietary supplement formulations in accordance with the present invention provide certain all-natural ingredients that support the maintenance of maximal levels of the “right” neurotransmitters for neuromuscular facilitation. The neurotransmitter of interest for the present invention is acetylcholine (Ach), which transmits messages to the skeletal muscles. As such, the neurotransmitter acetylcholine is necessary for the neuromuscular facilitation process to work properly. Dramatic improvements in motor skills and cognitive functions are produced by an increase in the neurotransmitter acetylcholine by means of dietary supplementation.

In further detail, neurotransmitters are stored in the neuron's bulbous end (axon), and when an electrical impulse traveling along the nerve reaches the axon, the neurotransmitter is released and travels across the synapse (the junction that separates one neuron from another neuron or a muscle), either promoting or inhibiting continued electrical impulses along the nerve. The specific type of neuron involved with the neuromuscular facilitation process is a motor neuron, which conveys electrical impulses from the central nervous system to a muscle. When a motor neuron “depolarizes,” an electrical current (i.e., the action potential) is passed down the nerve fiber. Upon reaching the end of the neuron, the impulse causes the release of the neurotransmitter acetylcholine. Upon release, the acetylcholine binds to acetylcholine receptors on the targeted striated muscle fibers that are in close proximity to the neuron (i.e., the motor end plate). The binding of acetylcholine to the muscle membrane initiates an action potential, which causes an electric current to pass onto the muscle membrane, thereby stimulating muscle contraction. After acetylcholine stimulates a muscle tissue, a special enzyme, acetylcholinesterase (AchE), quickly degrades acetylcholine into acetate and choline, which are absorbed back into the first neuron to be recycled into acetylcholine molecule again.

There are other numerous factors—other than increasing acetylcholine levels within the body—that contribute to the overall effects of the dietary nutritional composition supplement in accordance with the present invention. For example, one important aspect of the present invention is its ability to properly maintain the neural cell membrane by providing cytidine 5′-diphosphocholine (CDP-choline)—a naturally-occurring precursor to one of the most important phospholipids, phosphatidylcholine—as one of the key ingredients of the formulation in accordance with the present invention. A principal area of current scientific research concerns the membrane that surrounds the neuronal cell and the role that this membrane plays in ensuring proper function of the neuron as part of the central nervous system and the peripheral nervous system. Above all, the function of this neural cell membrane is to protect the cell; but, it also coordinates a high level of neuronal (i.e., neuron to neuron) and neuromuscular (i.e., neuron to muscle tissue) communication, which, in turn, contributes to improved neuromuscular facilitation.

Phospholipids—the various fatty-acid mineral molecules that comprise a significant portion of the neural cell membrane—play key roles in maintaining neural cell efficiency. Phospholipids are the attendants at the entry gates into our neural cells and are involved in transporting certain substrates, such as fuel, that are needed for healthy and efficient cell operation. Phospholipids help maintain and regulate cell membrane integrity, strength, permeability, elasticity, and resistance to stress, among other functions. Lack of sufficient nutrients to properly maintain phospholipids would interfere with the proper maintenance and regulation of neural cell membranes in the brain, interfering with an individual's ability to think clearly, focus and maintain attention, which would, in turn, further interfere with muscle memory and cognitive functions. As such, supporting proper levels of phospholipids by means of dietary supplementation is one important aspect of the present invention.

Another important aspect of the present invention is its ability to neutralize and inhibit certain neurotoxins by means of dietary supplementation, thereby improving cognitive functions such as memory and mental focus. It is widely known that many neurotoxins—certain degenerative chemicals that damage or destroy the brain and other nerve tissues—are factors in brain-motor degenerative diseases such as Parkinson's, Alzheimer's and Huntington's diseases. As such, neurotoxins directly interfere with the muscle memory process and cognitive functions such as maintenance of mental focus.

Amyloid beta is one such major neurotoxin. Amyloid beta is formed in part through oxidative damage caused by free radicals, and it causes further free radical production, causing intracellular oxidative damage. The neurotoxic effect of amyloid beta occurs primarily in the cholinergic neurons (those neurons for which acetylcholine is the neurotransmitter) in certain regions of the brain, notably the hippocampus, which governs vital aspects of the neuromuscular facilitation process and cognitive functions.

There is also an association between elevated homocysteine levels in the blood and impaired cognitive performance. The dietary nutritional composition supplement in accordance with the present invention provides specific nutrients that reduce levels of the neurotoxin homocysteine. One of these components is a group of polyphenols known as cucurminoids that are found in the anti-inflammatory spice turmeric and catechins in green tea. In further detail, homocysteine is chemically transformed into methionine and cysteine with the help of folic acid, vitamin B₁₂, and vitamin B₆. Therefore, insufficient amounts of these vitamins in the body can hamper the natural breakdown of homocysteine. This can cause homocysteine to accumulate in the blood. Conversely, a higher folic acid intake and higher levels of body folate are associated with lower blood homocysteine levels. Accordingly, certain embodiments of the present invention include folic acid, vitamin B₁₂, and vitamin B₆ as part of its formulation.

In sum, the various embodiments of the present invention are directed to establishing and maintaining high levels of acetylcholine, by means of administration of various combinations of natural ingredients that have various synergistic effects on acetylcholine metabolism within the body, and reducing amyloid beta and elevated homocysteine levels, thereby resulting in maximum enhancement of neuromuscular facilitation and cognitive functions.

To date, there is no known safe, all-natural, dietary supplement that specifically enhances muscle memory and cognitive functions by supporting the following functions within the body: (1) increases acetylcholine levels within the body, thereby improving neuromuscular facilitation; (2) inhibiting the action of acetylcholinesterase, the enzyme that catalyzes the breakdown of acetylcholine, thereby improving neurotransmission communication processes; (3) reduces amyloid beta toxicity in the brain; (4) reduces blood levels of neurotoxin homocysteine; (5) supports the overall neural cell membrane structure; (6) provides methyl donors to support the proper metabolism of the neurotransmitter acetylcholine and the breakdown of homocysteine; and (7) increases serotonin and norepinephrine in the brain to provide calm mental energy with positive mood impact.

In view of the above, there exists a need for a dietary nutrional composition supplement that addresses all the issues associated with the entire neurotransmission process, as set forth above, and safely and effectively enhances muscle memory and cognitive functions, while at the same time providing calm mental energy with positive mood impact. The combination of the aforementioned synergistic effects of neuromuscular facilitation, mental focus and calm mental energy are the critical attributes for any athletic endeavor that requires a high degree of physical accuracy and intense mental concentration, such as golfing and archery.

### DETAILED DESCRIPTION OF THE INVENTION

A primary embodiment of the present invention is a method and composition for promoting neuromuscular facilitation and cognitive functions by orally administering effective amounts of: (1) choline, which may be selected from one or more of the group consisting of choline bitartrate, choline citrate, choline chloride, naturally occurring choline or a derivative or metabolite thereof, and synthetic choline; and (2) dimethylaminoethanol (DMAE), which may be selected from one or more of the group consisting of naturally occurring DMAE or a derivative or metabolite thereof and synthetic DMAE.

Choline is a precursor of acetylcholine and phosphatidylcholine, the significance of which in neuromuscular facilitation is discussed above. Choline is a dietary component essential for normal function of all cells. Choline and its metabolites are needed for three main physiological purposes:
It directly affects cholinergic neurotransmission. That is, choline is a metabolic precursor to acetylcholine (i.e., acetylcholine synthesis), and acetylcholine is the neurotransmitter responsible for neuron-to-muscle transmission of information.
It assures the structural integrity and signaling roles for neural cell membranes.
It is a major source of methyl-groups in the diet. That is, one of choline's metabolites, betaine, participates in the methylation of homocysteine to form methionine. As discussed above, chemical transformation of homocysteine into methionine and cysteine reduces blood homocysteine levels.

The amount of choline included in various formulations in accordance with the present invention is specifically controlled to maximize the physiological functions of acetylcholine as discussed above, which takes into consideration its interactions with other factors involved in improving neuromuscular facilitation and cognitive functions.

Dimethylaminoethanol (DMAE) is a chemical compound related to choline. It is a precursor to the neurotransmitter acetylcholine. It is believed that DMAE is methylated to choline in the brain. As such, dietary supplementation of DMAE supports acetylcholine synthesis.

In addition, dietary supplementation of DMAE is reported to have nootropic effects, such as elevating mood, improving memory and learning, heightening intelligence, increasing physical energy, and, in laboratory animals, extending the life span of animals. DMAE has other beneficial effects, such as decreasing fatigue during the day, promoting sounder sleep at night, as well as needing less sleep when taking DMAE. In addition, DMAE further facilitates cell-to-cell communication in a way similar to that of acetylcholine, thereby improving neuromuscular facilitation.

In achieving these positive benefits, DMAE works by accelerating the brain's synthesis and turnover of the neurotransmitter acetylcholine, which, in turn, plays a key role in maximizing muscle memory. DMAE may also work, in part, by inhibiting choline metabolism in peripheral tissues, thereby increasing body choline levels by causing free choline to accumulate in blood, enter the brain, and stimulate cholinergic receptors.

In a further embodiment, the present invention is comprised of a dietary nutritional supplement composition, and a method for the administration thereof, for promoting neuromuscular facilitation and for enhancing cognitive functions, wherein the composition is comprised of: (1) an effective amount of choline, as set forth above; (2) an effective amount of DMAE, as set forth above; and (3) an effective amount of cytidine 5′-diphosphocholine (CDP-choline), which may be selected from one or more of the group consisting of naturally occurring CDP-choline, or a derivative or metabolite thereof, and synthetic CDP-choline.

As discussed above, CDP-choline is a naturally occurring precursor to one of the most important phospholipids, phosphatidylcholine, that is a major component of cellular membranes and functions in the transport of lipoproteins in tissues. CDP-choline also acts as a neuroprotector. Specifically, neurons are more stable when CDP-choline is present, thereby supporting the conclusion that this increased stability during neuron-to-muscle communication can increase neuromuscular facilitation. In addition, CDP-choline improves motor performance and coordination in the rotorod and open field tests, thereby indicating that CDP-choline affects central mechanisms involved in cognitive behaviors, probably through a cholinergic action.

Therefore, dietary supplementation of CDP-choline levels greatly potentiates the neuromuscular facilitation and cognitive functions of the various formulations in accordance with the present invention.

In an even further embodiment, the present invention is comprised of a dietary nutritional supplement composition, and a method for the administration thereof, for promoting neuromuscular facilitation and enhancing cognitive functions, wherein the composition is comprised of: (1) an effective amount of choline, as set forth above; (2) an effective amount of DMAE, as set forth above; (3) an effective amount of CDP-choline, as set forth above; (4) an effective amount of turmeric extract, which is standardized to about 95% cucunminoids; and (5) an effective amount of decaffeinated green tea extract, which is standardized to about 95% polyphenols.

Turmeric extract and decaffeinated green tea extract are included in various formulations of the present invention for their ability to reduce amyloid beta toxicity.

Turmeric is a widely cultivated plant (Curcuma domestica and/or Curcuma longa) of India and its active ingredient is curcumin. Curcumin is a polyphenol and is known for its antitumor, antioxidant, anti-amyloid and anti-inflammatory properties. As such, turmeric and its curcuminoid content provides protection against the neurotoxin amyloid beta, thereby preventing damage to the cholinergenic nervous system, which further promotes neuromuscular facilitation and cognitive functions.

Another polyphenol compound that reduces amyloid beta toxicity is from green tea. The most important of the polyphenols in green tea are the catechins, and in particular, (−)-epigallocatechin-3-gallate (EGCG), a very strong antioxidant. EGCG from green tea extract also protects brains cells and other nerve cells from amyloid beta-induced toxicity.

The phenol groups in tea polyphenols are extremely active—easily able to capture and neutralize free radicals and other pro-oxidants. For example, EGCG from green tea is over 200 times more powerful than vitamin E in neutralizing pro-oxidants and free radicals that attack lipids (oils and fats). In addition, EGCG is also 20 times more potent than vitamin E in reducing oxidation. Green tea is essential for oxidation “clean up” in the brain and has been shown to reduce amyloid beta toxicity.

Of particular importance is the fact that green tea used in the dietary nutritional composition supplement in accordance with the present invention is a high-concentration EGCG, decaffeinated version. Many dietary supplements for increasing energy levels and promoting thermogenesis combine EGCG with caffeine. The present invention teaches the exact opposite. Using this standardized, decaffeinated version of green tea is important for the proper function of the dietary nutritional composition supplement in accordance with the present invention, because, while the neuroprotective benefits of EGCG are fully utilized, caffeine can induce anxiety, which can interfere with mental focus for accurate muscle memory.

In an even further embodiment, the present invention is comprised of a dietary nutritional supplement composition, and a method for the administration thereof, for promoting neuromuscular facilitation, and for enhancing cognitive functions, wherein the composition is comprised of: (1) an effective amount of choline, as set forth above; (2) an effective amount of DMAE, as set forth above; (3) an effective amount of CDP-choline, as set forth above; (4) an effective amount of turmeric extract, as set forth above; (5) an effective amount of decaffeinated green tea extract, as set forth above; (6) an effective amount of vitamin B₁, which may be selected from one or more of the group consisting of naturally occurring vitamin B₁ or a derivative or metabolite thereof and synthetic vitamin B₁; (7) an effective amount of vitamin B₅, which may be selected from one or more of the group consisting of naturally occurring vitamin B₅ or a derivative or metabolite thereof and synthetic vitamin B₅; (8) an effective amount of vitamin B₆, which may be selected from one or more of the group consisting of naturally occurring vitamin B₆ or a derivative or metabolite thereof and synthetic vitamin B₆; (9) an effective amount of vitamin B₁₂, which may be selected from one or more of the group consisting of naturally occurring vitamin B₁₂ or a derivative or metabolite thereof and synthetic vitamin B₁₂; (10) an effective amount of folic acid, which may be selected from one or more of the group consisting of naturally occurring folic acid or a derivative or metabolite thereof and synthetic folic acid; and (11) an effective amount of dimethylglycine (DMG), which may be selected from one or more of the group consisting of naturally occurring DMG or a derivative or metabolite thereof and synthetic DMG.

Studies have found an association between elevated levels of homocysteine, impaired cognitive performance and dementia. Most elevations in homocysteine result from inadequate folate, vitamin B₁₂, or vitamin B₆ intake. Low intake of B vitamins and high blood homocysteine levels predict cognitive decline and that supplementation with vitamin B₆, vitamin B₁₂, folic acid, and DMG (betaine) helps to reduce blood homocysteine levels, thereby improving cognitive functions. In addition, folic acid supplementation increases levels of betaine and decreases levels of homocysteine, thereby improving cognitive functions. Therefore, by including vitamin B₆, vitamin B₁₂, folic acid, and DMG (betaine)—and possibly vitamin B₁ and vitamin B₅—in various formulations in accordance with the present invention, the neuron system is better protected, thereby preserving levels of acetylcholine even more.

DMG, as one of the nutrients in various formulations in accordance with the present invention, has a two-fold benefit as it not only lowers the neurotoxin homocysteine, it also provides methyl donors critical to the methylation process. A methyl donor is any substance that can transfer a methyl group to another substance (in a process called methylation), and many important biochemical processes rely on methylation. Homocysteine is chemically transformed into methionine with the help of DMG (betaine), thereby lowering blood levels of homocysteine. As discussed above, maintaining normal homocysteine levels is important for the health of the cardiovascular system, and helps prevent strokes and possibly Alzheimer's disease. Moreover, methyl donors are important cofactors in the building blocks of neurotransmitters, specifically acetylcholine. That is, DMG (betaine) is an important source of methyl donors that is critical for the proper metabolism of the neurotransmitter acetylcholine.

As such, the various formulations in accordance with the present invention include the aforementioned B vitamins, folic acid, and DMG (betaine), to help reduce blood levels of homocysteine and to help with the overall metabolism of acetylcholine, thereby improving cognitive functions and neuromuscular facilitation.

In an even further embodiment, the present invention is comprised of a dietary nutritional supplement composition, and a method for the administration thereof, for promoting neuromuscular facilitation, and for enhancing cognitive functions, wherein the composition is comprised of: (1) an effective amount of choline, as set forth above; (2) an effective amount of DMAE, as set forth above; (3) an effective amount of CDP-choline, as set forth above; (4) an effective amount of turmeric extract, as set forth above; (5) an effective amount of decaffeinated green tea extract, as set forth above; (6) an effective amount of vitamin B₁, as set forth above; (7) an effective amount of vitamin B₅, as set forth above; (8) an effective amount of vitamin B₆, as set forth above; (9) an effective amount of vitamin B₁₂, as set forth above; (10) an effective amount of folic acid, as set forth above; (11) an effective amount of DMG, as set forth above; and (12) an effective amount of huperzine A.

As discussed above, after acetylcholine stimulates a muscle tissue, the enzyme acetylcholinesterase quickly degrades acetylcholine into acetate and choline, which are absorbed back into the first neuron to be recycled into acetylcholine molecule again. As such, increased physical activity results in the increased release of acetylcholine from the cholinergic nerve endings and the subsequent breakdown of acetylcholine through the increased activity of acetylcholinesterase, thereby resulting in depletion of acetylcholine for further stimulation of muscle tissue and impairment of the neuromuscular facilitation process.

Huperzine A, a naturally occurring sesquiterpene alkaloid found in the extracts of the firmoss Huperzia serrata, inhibits the action of the cholinesterase enzymes, such as acetylcholinesterase, thereby increasing the effective concentration of acetylcholine for the proper functioning of the neuromuscular facilitation process. That is, huperzine A is known as a potent inhibitor of the enzyme acetylcholinesterase. This is the same mechanism of action of pharmaceutical drugs such as galantamine and donepezil used to treat Alzheimer's disease. As such, huperzine A is currently being investigated as a possible treatment for diseases characterized by neurogeneration—particularly Alzheimer's disease.

As such, the various formulations in accordance with the present invention include huperzine A to increase acetylcholine levels and to counter the neurodegenerative effects of the Alzheimer's disease, thereby promoting the overall neuromuscular facilitation process and cognitive functions.

In an even further embodiment, the present invention is comprised of a dietary nutritional supplement composition, and a method for the administration thereof, for promoting neuromuscular facilitation, and for enhancing cognitive functions, wherein the composition is comprised of: (1) an effective amount of choline, as set forth above; (2) an effective amount of DMAE, as set forth above; (3) an effective amount of CDP-choline, as set forth above; (4) an effective amount of turmeric extract, as set forth above; (5) an effective amount of decaffeinated green tea extract, as set forth above; (6) an effective amount of vitamin B₁, as set forth above; (7) an effective amount of vitamin B₅, as set forth above; (8) an effective amount of vitamin B₆, as set forth above; (9) an effective amount of vitamin B₁₂, as set forth above; (10) an effective amount of folic acid, as set forth above; (11) an effective amount of DMG, as set forth above; (12) an effective amount of huperzine A, as set forth above; (13) an effective amount of Griffonia simplicifolia extract (about 95% 5-hydroxytriptophan (5-HTP)); and (14) an effective amount of 1-phenylalanine.

While increasing mental focus is one major aspect of the present invention, it is also important to ensure calm mental energy without producing anxiety. The present invention achieves this with two very important amino acids in various formulations in accordance with the present invention: 5-HTP and 1-phenylalanine.

5-HTP is a naturally occurring substance derived from the seeds of Griffonia simplicifolia, a West African medicinal plant. In humans, 5-HTP is the immediate nutrient precursor to the neurotransmitter serotonin (5-HT). This means that 5-HTP converts directly into serotonin in the brain. Serotonin is a specific neurotransmitter involved in promoting calm mood that can result in deliberate physical movements. Serotonin also has many important functions in the human body, including a role in sleep, appetite, memory, learning, temperature regulation, mood, and muscle contraction. Dietary supplementation of 5-HTP, thereby increasing serotonin levels in the brain, helps support healthy brain function. In addition, dietary supplementation with 5-HTP not only increases serotonin levels, it further helps to prevent anxiety and panic attacks.

An additional amino acid used in various formulations in accordance with the present invention is 1-phenylalanine. L-phenylalanine is used for increasing levels of norepinephrine, the neurotransmitter involved in producing mental energy and positive attitude. L-phenylalanine also successfully treats depression and amphetamine withdrawal because of its ability to increase the “feel good” amines in the brain. One such “feel good” amine is phenylethylamine, which is an important brain chemical that increases mood and positive outlook. This is especially important in the mental self-confidence aspect in any game of skill and achievement.

In sum, maximizing neuromuscular facilitation and cognitive functions is achieved by consuming various formulations in accordance with the present invention, which then induces the following biochemical reactions to take place in the body, and particularly, in the brain and at the muscle level:
Increases acetylcholine levels within the body.
Inhibits the action of acetylcholinesterase.
Reduces amyloid beta toxicity in the brain.
Reduces blood levels of neurotoxin homocysteine.
Supports the overall neural cell membrane structure.
Provides methyl donors to support the proper metabolism of the neurotransmitter acetylcholine and the breakdown of homocysteine.
Increases serotonin and norepinephrine in the brain to provide calm mental energy with positive mood impact.

### Optional Ingredients

The composition and method of the present invention for improving neuromuscular facilitation and enhancing cognitive functions, may optionally and additionally comprise further effective amounts of various ingredients (components, minerals, chemical formulations, derived chemical compounds, naturally occurring compounds and the like) as listed below. The optional ingredients (compounds and compositions) may be utilized with the combination of the ingredients as set forth above individually or in any combination. Furthermore, the optional ingredients listed may comprise both natural and synthetic formulations or derivations, and may comprise one or more of their derived sources.

Magnesium stearate—Optionally, the composition and method of the present invention may also include an effective amount of magnesium stearate, or a derivative or metabolite thereof, which is used in the dietary supplement industry as a lubricant to assist in processing the product on the machinery. The preferred machinery for processing the product in accordance with the invention is two-piece gelatin capsule machinery. However, the present invention is not limited to gelatin capsule; it may also include tablets forms, which may use magnesium stearate, stearic acid, microcrystalline cellulose, dicalcium phosphate, tricalcium phosphate, sodium starch glycolate, modified cellulose gum, vegetable stearine, etc.

Silicon dioxide—Optionally and additionally, the composition and method of the present invention may also include an effective amount of silicon dioxide, which is used as a flow agent. Silicone dioxide is known to ameliorate the effects of the hygroscopicity of the product ingredients. This ingredient is typically known in the industry as “amorphous” (i.e., it lacks crystalline structure) silica or silicon dioxide.

Nicotinamide adenine dinucleotide (NADH)—Optionally and additionally, the composition and method of the present invention may also include an effective amount of NADH, which may be selected from one or more of the group consisting of naturally occurring NADH or a derivative or metabolite thereof and synthetic NADH. NADH helps to regulate communication between nerve cells.

### Exemplary Compositions

The following example composition amounts can be utilized for both a composition and a method for improving neuromuscular facilitation and enhancing cognitive functions. These examples are not meant to limit the effective amounts and/or ranges of the ingredients and/or ranges of the ingredients and compounds contained within the present invention.

### EXAMPLE 1

A composition for improving neuromuscular facilitation, focus and learned behavior process, comprising:
about 5 mg to about 3,000 mg of choline;
about 10 mg to about 800 mg of DMAE;
about 5 mg to about 1,000 mg of CDP-choline; about 5 mg to about 1,000 mg of turmeric extract (about 95% cucurminoids);
about 20 mg to about 800 mg of green tea extract (about 95% polyphenols);
about 0.5 mg to about 200 mg of vitamin B₁;
about 0.5 mg to about 200 mg of vitamin B₅;
about 0.5 mg to about 200 mg of vitamin B₆;
about 100 mcg to about 2,000 mcg of vitamin B₁₂;
about 20 mcg to about 1,600 mcg of folic acid;
about 5 mg to about 1,000 mg of DMG, or in the alternative, about 5 mg to
about 1,000 mg of trimethylglycine;
about 5 mcg to about 10 mg of huperzine A;
about 10 mg to about 1,000 mg of Griffonia simplicifolia extract (about 95% 5-HTP), or, in the alternative, about 50 mg to about 2,000 mg of 1-tryptophan;
about 50 mg to about 1,500 mg of 1-phenylalanine, or in the alternative,
about 50 mg to about 1,500 mg of 1-tyrosine;
magnesium stearate; and
silicon dioxide,

Wherein the choline is selected from one or more of the group consisting of choline bitartrate, choline citrate, choline chloride, naturally occurring choline or a derivate or metabolite thereof, and synthetic choline;

wherein the DMAE is selected from one or more of the group consisting of naturally occurring DMAE, or a derivate or metabolite thereof, and synthetic DMAE;

wherein the CDP-choline is selected from one or more of the group consisting of naturally occurring CDP-choline, or a derivate or metabolite thereof, and synthetic CDP-choline;

wherein the turmeric extract is standardized to about 95% curcuminoids;

wherein the green tea extract is standardized to about 95% polyphenols;

wherein the vitamin B₁ is selected from one or more of the group consisting of naturally occurring vitamin B₁, or a derivate or metabolite thereof, and synthetic vitamin B₁;

wherein the vitamin B₅ is selected from one or more of the group consisting of naturally occurring vitamin B₅, or a derivate or metabolite thereof, and synthetic vitamin B₅;

wherein the vitamin B₆ is selected from one or more of the group consisting of naturally occurring vitamin B₆, or a derivate or metabolite thereof, and synthetic vitamin B₆;

wherein the vitamin B₁₂ is selected from one or more of the group consisting of naturally occurring vitamin B₁₂, or a derivate or metabolite thereof, and synthetic vitamin B₁₂;

wherein the folic acid is selected from one or more of the group consisting of naturally occurring folic acid, or a derivate or metabolite thereof, and synthetic folic acid;

wherein the DMG is selected from one or more of the group consisting of naturally occurring DMG, or a derivate or metabolite thereof, and synthetic DMG;

wherein the trimethylglycine is selected from one or more of the group consisting of naturally occurring trimethylglycine, or a derivate or metabolite thereof, and synthetic trimethylglycine;

wherein the huperzine A is selected from one or more of the group consisting of naturally occurring huperzine A, or a derivate or metabolite thereof, and synthetic huperzine A;

wherein the Griffonia simplicifolia extract is standardized to about 95% 5-HTP;

wherein the 1-tryptophan is selected from one or more of the group consisting of naturally occurring 1-tryptophan, or a derivate or metabolite thereof, and synthetic 1-tryptophan;

wherein the 1-phenylalanine is selected from one or more of the group consisting of naturally occurring 1-phenylalanine, or a derivate or metabolite thereof, and synthetic 1-phenylalanine;

wherein the 1-tyrosine is selected from one or more of the group consisting of naturally occurring 1-tyrosine, or a derivate or metabolite thereof, and synthetic 1-tyrosine; wherein the magnesium stearate is selected from one or more of the group consisting of naturally occurring magnesium stearate, or a derivate or metabolite thereof, and synthetic magnesium stearate; and

wherein the silicon dioxide is selected from one or more of the group consisting of naturally occurring silicon dioxide, or a derivate or metabolite thereof, and synthetic silicon dioxide.

The above example may be utilized in methods and compositions for improving neuromuscular facilitation, focus and learned behavior process.

### EXAMPLE 2

A composition for improving neuromuscular facilitation, focus and learned behavior process, comprising:
about 10 mg to about 1,500 mg of choline;
about 20 mg to about 400 mg of DMAE;
about 10 mg to about 500 mg of CDP-choline;
about 10 mg to about 500 mg of turmeric extract (about 95% cucurminoids);
about 40 mg to about 400 mg of green tea extract (about 95% polyphenols);
about 1.0 mg to about 100 mg of vitamin B₁;
about 1.0 mg to about 100 mg of vitamin B₅;
about 1.0 mg to about 100 mg of vitamin B₆;
about 200 mcg to about 1,000 mcg of vitamin B₁₂;
about 40 mcg to about 800 mcg of folic acid;
about 10 mg to about 500 mg of DMG, or in the alternative, about 10 mg to
about 500 mg of trimethylglycine;
about 10 mcg to about 5 mg of huperzine A;
about 20 mg to about 500 mg of Griffonia simplicifolia extract (about 95% 5-.HTP), or, in the alternative, about 100 mg to about 1,000 mg of 1-tryptophan;
about 100 mg to about 750 mg of 1-phenylalanine, or in the alternative, about 100 mg to about 750 mg of 1-tyrosine;
magnesium stearate; and
silicon dioxide.

The sources of each ingredient and compound as set forth in Example 1 is also applicable to Example 2, which also applies to methods and compositions for improving neuromuscular facilitation, focus and learned behavior process.

### EXAMPLE 3

A composition for improving neuromuscular facilitation and cognitive functions, comprising:
about 175 mg to about 350 mg of choline;
about 40 mg to about 80 mg of DMAE;
about 75 mg to about 150 mg of CDP-choline;
about 65 mg to about 130 mg of turmeric extract (about 95% cucurnainoids);
about 100 mg to about 200 mg of green tea extract (about 95% polyphenols);
about 8.5 mg to about 17 mg of vitamin B₁;
about 43 mg to about 86 mg of vitamin B₅;
about 10 mg to about 20 mg of vitamin B₆;
about 450 mcg to about 900 mcg of vitamin B₁₂;
about 400 mcg to about 800 mcg of folic acid;
about 40 mg to about 80 mg of DMG, or in the alternative, about 50 mg to
about 100 mg of trimethylglycine;
about 50 mcg to about 100 mcg of huperzine A;
about 25 mg to about 50 mg of Griffonia simplicifolia extract (about 95% 5-HTP), or, in the alternative, about 100 mg to about 200 mg of 1-tryptophan;
about 130 mg to about 260 mg of 1-phenylalanine, or in the alternative, about 200 mg to about 400 mg of 1-tyrosine;
magnesium stearate; and
silicon dioxide.

The sources of each ingredient and compound as set forth in Example 1 is also applicable to Example 3, which also applies to methods and compositions for improving neuromuscular facilitation, focus and learned behavior process.

### Forms of Composition

The above combination of ingredients may be administered orally, through an oral preparation, or in any other manner as consistent with those methods presently existing for administration of other dietary nutritional supplements, as well as the methods of the present invention for improving neuromuscular facilitation and enhancing cognitive functions. The oral preparation of the above combination of ingredients comprises tablets, capsules, gelatin capsules (e.g., two-piece gelcaps), or powder. The composition as a powder may simply be incorporated into a food. The powder form of the composition may also be used as drink mixes, or as an ingredient to create a drink, which may include flavors and organic acids, colors, etc. The composition as a tablet, capsule, gelatin capsule or gelcap may also contain at least one pharmaceutically acceptable carrier. The pharmaceutically acceptable carrier may be selected from one or more of the group consisting of a binding agent, filler, lubricant, disintegrant, or a wetting agent, as these terms are commonly known to those skilled in the art. Furthermore, the oral preparation of the above ingredients may also comprise a liquid suspension, colloidal suspension, shake or aqueous mixture.

### Times

The combination of the ingredients set forth in each example above may be administered one or more times per day, depending upon the effective dosage amount utilized as described in the preceding paragraphs. For example, the daily doses as set forth in each example may be orally administered once daily. Optionally, and more preferably, the dosage amounts are divided and taken at various times during the day, again depending upon the dosages. As such, the combination of the ingredients set forth in each example above, wherein the daily dosages as set forth in each paragraph are reduced by one-half (50%) and each one-half dose of the combination of the ingredients is orally administered twice (2) daily. Ideal times for the twice daily dosage may be just before breakfast and just before lunch. For example, the combination of the ingredients set forth in each example above, wherein the daily doses as set forth in each example are reduced by one-half (50%) and each one-half (½) dose of the combination of the ingredients is orally administered twice daily as follows: the first one-half (½) daily dose is orally administered before breakfast and the second one-half (½) daily dose is orally administered at mid-afternoon and before lunch.

Other dosage amounts and times, depending upon the number of subdivisions of the dosages and/or times of day can be utilized in administering the present invention. For example, the combination of the ingredients as set forth in each example above, wherein the daily doses as set forth in each example are reduced by one-third (⅓) and each one-third (⅓) dose of the combination of the ingredients is orally administered three (3) times daily.

It should be understood that the specific formulations and methods described herein are exemplary and should not be construed to limit the invention, which will be defined solely by the claims below. Further, those skilled in the art may now make numerous uses and modifications of the specific embodiments described, without departing from the inventive concepts. Also, the invention contemplates that formulations in accordance therewith may be made with combinations of the disclosed ingredients other than those described above as long as they are within the scope of the claims below. There are many other variations of clinical and metabolic situations, specific methods of addressing such situations, and formulations and compositions that can be included in a document such as this. Consequently, the invention is to be construed as embracing each and every novel feature and novel combination of features present in and/or possessed by the compositions and methods described and claimed by their equivalents.

Although some products nutritionally support specific biochemical reactions that parallel certain aspects of neuromuscular facilitation and cognitive functions, no previously known dietary supplement has provided in a single formulation the wide range of therapeutic benefits that are provided by the present invention. All components have been included in the present invention at known therapeutically effective amounts in order to provide broad-spectrum therapeutic benefits with minimal side effects.

In addition, having all components available in a single formulation provides cost savings for the user and more efficient treatment protocols by healthcare professionals.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims.

The present invention has been described in what are considered to be the most practical and preferred embodiments. It is anticipated, however, that departures may be made therefrom and that obvious modifications will be implemented by those skilled in the art. It will be appreciated that those skilled in the art will be able to devise numerous arrangements and various which, although not explicitly shown or described herein, embody the principles of the invention and are within their spirit and scope.

It is intended that all modifications and variations be included within the scope of the invention. The claims are meant to cover the claimed components and steps in any arrangement or sequence which is effective to meet the objectives intended for the invention, unless the context specifically indicates the contrary. It is important, therefore, that the claims be regarded as including such equivalent compositions insofar as they do not depart from the spirit and scope of the present invention.

Further, the purpose of the foregoing description and abstract is to enable the U.S. Patent and Trademark Office and the public generally, and especially the scientists, technicians and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

What is claimed is:

1. A composition for enhancing neuromuscular facilitation and cognitive functions, comprising effective amounts of choline, dimethylaminoethanol, and cytidine 5′-diphosphocholine.

2. The composition of claim 1 wherein:
the effective amount of choline is about 5 mg to about 3,000 mg;
the effective amount of dimethylaminoethanol is about 10 mg to about 800 mg; and
the effective amount of cytidine 5′-diphosphocholine is about 5 mg to about 1,000 mg.

3. The composition of claim 1 wherein:
the effective amount of choline is about 10 mg to about 1,500 mg;
the effective amount of dimethylaminoethanol is about 20 mg to about 400 mg; and
the effective amount of cytidine 5′-diphosphocholine is about 10 mg to about 500 mg.

4. The composition of claim 1 wherein:
the effective amount of choline is about 175 mg to about 350 mg;
the effective amount of dimethylaminoethanol is about 40 mg to about 80 mg; and
the effective amount of cytidine 5′-diphosphocholine is about 75 mg to about 150 mg.

5. The composition of claim 1, further comprising effective amounts of turmeric extract and decaffeinated green tea extract.

6. The composition of claim 5 wherein:
the effective amount of choline is about 5 mg to about 3,000 mg;
the effective amount of dimethylaminoethanol is about 10 mg to about 800 mg;
the effective amount of cytidine 5′-diphosphocholine is about 5 mg to about 1,000 mg.
the effective amount of turmeric extract is about 5 mg to about 1,000 mg; and
the effective amount of decaffeinated green tea extract is about 20 mg to about 800 mg.

7. The composition of claim 5 wherein:
the effective amount of choline is about 10 mg to about 1,500 mg;
the effective amount of dimethylaminoethanol is about 20 mg to about 400 mg;
the effective amount of cytidine 5′-diphosphocholine is about 10 mg to about 500 mg;
the effective amount of turmeric extract is about 10 mg to about 500 mg; and
the effective amount of decaffeinated green tea extract is about 40 mg to about 400 mg.

8. The composition of claim 5 wherein:
the effective amount of choline is about 175 mg to about 350 mg;
the effective amount of dimethylaminoethanol is about 40 mg to about 80 mg;
the effective amount of cytidine 5′-diphosphocholine is about 75 mg to about 150 mg;
the effective amount of turmeric extract is about 65 mg to about 130 mg; and
the effective amount of decaffeinated green tea extract is about 100 mg to about 200 mg.

9. The composition of claim 5, further comprising effective amounts of vitamin B₁, vitamin B₅, vitamin B₆, vitamin B₁₂, folic acid, and dimethylglycine.

10. The composition of claim 9 wherein:
the effective amount of choline is about 5 mg to about 3,000 mg;
the effective amount of dimethylaminoethanol is about 10 mg to about 800 mg;
the effective amount of cytidine 5′-diphosphocholine is about 5 mg to about 1,000 mg;
the effective amount of turmeric extract is about 5 mg to about 1,000 mg;
the effective amount of decaffeinated green tea extract is about 20 mg to about 800 mg;
the effective amount of vitamin B₁ is about 0.5 mg to about 200 mg;
the effective amount of vitamin B₅ is about 0.5 mg to about 200 mg;
the effective amount of vitamin B₆ is about 0.5 mg to about 200 mg;
the effective amount of vitamin B₁₂ is about 0.1 mg to about 2.0 mg;
the effective amount of folic acid is about 20 mcg to about 1,600 mcg; and
the effective amount of dimethylglycine is about 5 mg to about 1,000 mg.

11. The composition of claim 9 wherein:
the effective amount of choline is about 10 mg to about 1,500 mg;
the effective amount of dimethylaminoethanol is about 20 mg to about 400 mg;
the effective amount of cytidine 5′-diphosphocholine is about 10 mg to about 500 mg;
the effective amount of turmeric extract is about 10 mg to about 500 mg;
the effective amount of decaffeinated green tea extract is about 40 mg to about 400 mg;
the effective amount of vitamin B₁ is about 1.0 mg to about 100 mg;
the effective amount of vitamin B₅ is about 1.0 mg to about 100 mg;
the effective amount of vitamin B₆ is about 1.0 mg to about 100 mg;
the effective amount of vitamin B₁₂ is about 0.2 mg to about 1.0 mg;
the effective amount of folic acid is about 40 mcg to about 800 mcg; and
the effective amount of dimethylglycine is about 10 mg to about 500 mg.

12. The composition of claim 9 wherein:
the effective amount of choline is about 175 mg to about 350 mg;
the effective amount of dimethylaminoethanol is about 40 mg to about 80 mg;
the effective amount of cytidine 5′-diphosphocholine is about 75 mg to about 150 mg;
the effective amount of turmeric extract is about 65 mg to about 130 mg;
the effective amount of decaffeinated green tea extract is about 100 mg to about 200 mg;
the effective amount of vitamin B₁ is about 8.5 mg to about 17 mg;
the effective amount of vitamin B₅ is about 43 mg to about 86 mg;
the effective amount of vitamin B₆ is about 10 mg to about 20 mg;
the effective amount of vitamin B₁₂ is about 450 mcg to about 900 mcg;
the effective amount of folic acid is about 400 mcg to about 800 mcg; and
the effective amount of dimethylglycine is about 40 mg to about 80 mg.

13. The composition of claim 9, further comprising an effective amount of huperzine A.

14. The composition of claim 13 wherein:
the effective amount of choline is about 5 mg to about 3,000 mg;
the effective amount of dimethylaminoethanol is about 10 mg to about 800 mg;
the effective amount of cytidine 5′-diphosphocholine is about 5 mg to about 1,000 mg;
the effective amount of turmeric extract is about 5 mg to about 1,000 mg;
the effective amount of decaffeinated green tea extract is about 20 mg to about 800 mg;
the effective amount of vitamin B₁ is about 0.5 mg to about 200 mg;
the effective amount of vitamin B₅ is about 0.5 mg to about 200 mg;
the effective amount of vitamin B₆ is about 0.5 mg to about 200 mg;
the effective amount of vitamin B₁₂ is about 0.1 mg to about 2.0 mg;
the effective amount of folic acid is about 20 mcg to about 1,600 mcg;
the effective amount of dimethylglycine is about 5 mg to about 1,000 mg; and
the effective amount of huperzine A is about 5 mcg to about 10 mg.

15. The composition of claim 13 wherein:
the effective amount of choline is about 10 mg to about 1,500 mg;
the effective amount of dimethylaminoethanol is about 20 mg to about 400 mg;
the effective amount of cytidine 5′-diphosphocholine is about 10 mg to about 500 mg;
the effective amount of turmeric extract is about 10 mg to about 500 mg;
the effective amount of decaffeinated green tea extract is about 40 mg to about 400 mg;
the effective amount of vitamin B₁ is about 1.0 mg to about 100 mg;
the effective amount of vitamin B₅ is about 1.0 mg to about 100 mg;
the effective amount of vitamin B₆ is about 1.0 mg to about 100 mg;
the effective amount of vitamin B₁₂ is about 0.2 mg to about 1.0 mg;
the effective amount of folic acid is about 40 mcg to about 800 mcg;
the effective amount of dimethylglycine is about 10 mg to about 500 mg; and
the effective amount of huperzine A is about 10 mcg to about 5 mg.

16. The composition of claim 13 wherein:
the effective amount of choline is about 175 mg to about 350 mg;
the effective amount of dimethylaminoethanol is about 40 mg to about 80 mg;
the effective amount of cytidine 5′-diphosphocholine is about 75 mg to about 150 mg;
the effective amount of turmeric extract is about 65 mg to about 130 mg;
the effective amount of decaffeinated green tea extract is about 100 mg to about 200 mg; the effective amount of vitamin B₁ is about 8.5 mg to about 17 mg;
the effective amount of vitamin B₅ is about 43 mg to about 86 mg;
the effective amount of vitamin B₆ is about 10 mg to about 20 mg;
the effective amount of vitamin B₁₂ is about 450 mcg to about 900 mcg;
the effective amount of folic acid is about 400 mcg to about 800 mcg;
the effective amount of dimethylglycine is about 40 mg to about 80 mg; and
the effective amount of huperzine A is about 50 mcg to about 100 mcg.

17. The composition of claim 13, further comprising effective amounts of Griffonia simplicifolia extract and 1-phenylalanine.

18. The composition of claim 17 wherein:
the effective amount of choline is about 5 mg to about 3,000 mg;
the effective amount of dimethylaminoethanol is about 10 mg to about 800 mg;
the effective amount of cytidine 5′-diphosphocholine is about 5 mg to about 1,000 mg;
the effective amount of turmeric extract is about 5 mg to about 1,000 mg;
the effective amount of decaffeinated green tea extract is about 20 mg to about 800 mg;
the effective amount of vitamin B₁ is about 0.5 mg to about 200 mg;
the effective amount of vitamin B₅ is about 0.5 mg to about 200 mg;
the effective amount of vitamin B₆ is about 0.5 mg to about 200 mg;
the effective amount of vitamin B₁₂ is about 0.1 mg to about 2.0 mg;
the effective amount of folic acid is about 20 mcg to about 1,600 mcg;
the effective amount of dimethylglycine is about 5 mg to about 1,000 mg;
the effective amount of huperzine A is about 5 mcg to about 10 mg;
the effective amount of Griffonia simplicifolia extract is about 10 mg to about 1,000 mg; and
the effective amount of 1-phenylalanine is about 50 mg to about 1,500 mg.

19. The composition of claim 17 wherein:
the effective amount of choline is about 10 mg to about 1,500 mg;
the effective amount of dimethylaminoethanol is about 20 mg to about 400 mg;
the effective amount of cytidine 5′-diphosphocholine is about 10 mg to about 500 mg;
the effective amount of turmeric extract is about 10 mg to about 500 mg;
the effective amount of decaffeinated green tea extract is about 40 mg to about 400 mg;
the effective amount of vitamin B₁ is about 1.0 mg to about 100 mg;
the effective amount of vitamin B₅ is about 1.0 mg to about 100 mg;
the effective amount of vitamin B₆ is about 1.0 mg to about 100 mg;
the effective amount of vitamin B₁₂ is about 0.2 mg to about 1.0 mg;
the effective amount of folic acid is about 40 mcg to about 800 mcg;
the effective amount of dimethylglycine is about 10 mg to about 500 mg;
the effective amount of huperzine A is about 10 mcg to about 5 mg;
the effective amount of Griffonia simplicifolia extract is about 20 mg to about 500 mg; and
the effective amount of 1-phenylalanine is about 100 mg to about 750 mg.

20. The composition of claim 17 wherein:
the effective amount of choline is about 175 mg to about 350 mg;
the effective amount of dimethylaminoethanol is about 40 mg to about 80 mg;
the effective amount of cytidine 5′-diphosphocholine is about 75 mg to about 150 mg;
the effective amount of turmeric extract is about 65 mg to about 130 mg;
the effective amount of decaffeinated green tea extract is about 100 mg to about 200 mg;
the effective amount of vitamin B₁ is about 8.5 mg to about 17 mg;
the effective amount of vitamin B₅ is about 43 mg to about 86 mg;
the effective amount of vitamin B₆ is about 10 mg to about 20 mg;
the effective amount of vitamin B₁₂ is about 450 mcg to about 900 mcg;
the effective amount of folic acid is about 400 mcg to about 800 mcg;
the effective amount of dimethylglycine is about 40 mg to about 80 mg;
the effective amount of huperzine A is about 50 mcg to about 100 mcg;
the effective amount of Griffonia simplicifolia extract is about 25 mg to about 50 mg; and
the effective amount of 1-phenylalanine is about 130 mg to about 260 mg.

21. A composition for enhancing neuromuscular facilitation and cognitive functions, comprising:
about 175 mg of choline;
about 40 mg of dimethylaminoethanol;
about 75 mg of cytidine 5′-diphosphocholine;
about 65 mg of turmeric extract;
about 100 mg of decaffeinated green tea extract;
about 8.5 mg of vitamin B₁;
about 43 mg of vitamin B₅;
about 10 mg of vitamin B₆;
about 450 mcg of vitamin B₁₂;
about 400 mcg of folic acid;
about 40 mg of dimethylglycine;
about 50 mcg of huperzine A;
about 25 mg of Griffonia simplicifolia extract; and
about 130 mg of 1-phenylalanine.

22. The composition of claim 21 wherein:
the choline is selected from one or more of the group consisting of choline bitartrate, choline citrate, choline chloride, naturally occurring choline or a derivative or metabolite thereof, and synthetic choline;
the dimethylaminoethanol is selected from one or more of the group consisting of naturally occurring dimethylaminoethanol or a derivative or metabolite thereof and synthetic dimethylaminoethanol;
the cytidine 5′-diphosphocholine is selected from one or more of the group consisting of naturally occurring cytidine 5′-diphosphocholine or a derivative or metabolite thereof and synthetic cytidine 5′-diphosphocholine;
the turmeric extract is standardized to about 95% cucurminoids;
the decaffeinated green tea extract is standardized to about 95% polyphenols;
the vitamin B₁ is selected from one or more of the group consisting of naturally occurring vitamin B₁ or a derivative or metabolite thereof and synthetic vitamin B₁;
the vitamin B₅ is selected from one or more of the group consisting of naturally occurring vitamin B₅ or a derivative or metabolite thereof and synthetic vitamin B₅;
the vitamin B₆ is selected from one or more of the group consisting of naturally occurring vitamin B₆ or a derivative or metabolite thereof and synthetic vitamin B₆;
the vitamin B₁₂ is selected from one or more of the group consisting of naturally occurring vitamin B₁₂ or a derivative or metabolite thereof and synthetic vitamin B₁₂;
the folic acid is selected from one or more of the group consisting of naturally occurring folic acid or a derivative or metabolite thereof and synthetic folic acid;
the dimethylglycine is selected from one or more of the group consisting of naturally occurring dimethylglycine or a derivative or metabolite thereof and synthetic dimethylglycine;
the huperzine A is selected from one or more of the group consisting of naturally occurring huperzine A or a derivative or metabolite thereof and synthetic Huperzine A;
the Griffonia simplicifolia extract is standardized to about 95% 5-hydroxytriptophan; and
the 1-phenylalanine is selected from one or more of the group consisting of naturally occurring 1-phenylalanine or a derivative or metabolite thereof and synthetic 1-phenylalanine.
